(19) United States
(12) Patent Application Publication
(10) Pub. No.: US 2011/0244594 A1
(43) Pub. Date: Oct. 6, 2011

(54) TARGET SUBSTANCE DETECTING METHOD AND TARGET SUBSTANCE DETECTING APPARATUS

(75) Inventor: Kazuyoshi HORII, Kanagawa-ken (JP)

(73) Assignee: FUJIFILM CORPORATION, Tokyo (JP)

(21) Appl. No.: 13/045,358

(22) Filed: Mar. 10, 2011

(30) Foreign Application Priority Data
Mar. 31, 2010 (JP) 083358/2010

(51) Int. Cl. G01N 33/53; C12M 1/42

(52) U.S. Cl. 436/501; 435/287.1

(57) ABSTRACT

A target substance detecting method employs a detecting chip equipped with a fine flow channel. Examinations, in which clogging of the flow channel and inhibition of immune reactions can be easily and expediently prevented without preliminary processes, are enabled to be performed without fluctuations in hemolysis rates. The detecting chip having a flow channel base having the flow channel and a detecting portion formed in the flow channel is employed. A liquid sample that may contain a target substance and contains cellular non target substances is caused to flow into the flow channel. An ultrasonic wave emitting section provided upstream of the detecting portion emits ultrasonic waves into the liquid sample from a direction perpendicular to the longitudinal direction of the flow channel such that a standing wave is generated within the flow channel.

### BACKGROUND OF THE INVENTION

1. Field of the Invention

The present invention is related to a target substance detecting method and a target substance detecting apparatus that employ a detecting chip equipped with a fine flow channel.

2. Description of the Related Art

Recently, biochemical examinations (immunological examinations) that utilize immune reactions employing detecting chips equipped with fine flow channels on the micron level formed by fine processing techniques are being researched. By miniaturizing the flow channels, the amounts of samples to be collected from organisms can be reduced. In addition, the apparatus that includes the detecting chip can be miniaturized as a whole. Further, by miniaturizing the apparatus that includes the detecting chip as a whole, it becomes possible to realize POCT (Point Of Care Testing) in clinics and homes. Immunological examinations are widely utilized for various diagnoses and observations, and are an important method in clinical examinations. Presently, various immunological apparatuses have been developed, and high level analysis, such as simultaneous examination of a plurality of examination items, is possible.

In immunological examinations, blood is generally employed as test samples. Blood is constituted by blood cell components (also referred to as cellular components, which include red blood cells, white blood cells, and platelets) and plasma components (also referred to as fluid components, and refer to plasma or blood serum, which is plasma from which coagulants have been removed). It is often the case that plasma is used as a test sample.

In the case that plasma components are employed as a test sample and examination is performed using a detecting chip such as that described above, a preliminary process, in which blood cell components having greater specific weights than plasma components are centrifugally separated from collected blood, is performed. This is because the sizes of red blood cells (96% of blood cell components), white blood cells (3%), and platelets (1%) that constitute blood cell components of human blood are approximately 8 μm, approximately 6 μm to 22 μm, and approximately 1 μm to 4 μm, respectively. These sizes are large with respect to the flow channel. Therefore, when blood testing is performed using the detecting chip described above, problems, such as the blood cell components (particularly red blood cells) causing clogging of the flow channel and inhibiting immune reactions, occur. However, in the case that such a preliminary process is performed, other problems, such as a centrifugal separating apparatus being necessary, a long period of time being required for the centrifugal separation process due to the slight difference in the specific weights of blood cell components and plasma components, and the number of processing steps increasing, occur.

Meanwhile, an example of a method that separates blood cells without the need for such a preliminary process is disclosed in International Patent Publication No. WO98/08606. In this method, a blood cell separating film is provided in an upstream portion of the flow channel, and blood cell components are separated simply by spotting blood. However, although this method that employs the blood cell separating film can solve the problem of clogging of the flow channel, the possibility of the blood cell separating film becoming clogged exists. Such clogging may result in problems, such as an insufficient amount of a test sample flowing into the flow channel, and expedient measurements being inhibited. It is possible to employ a pump or the like to apply positive or negative pressure in order to accelerate blood cell separation using the blood cell separating film. However, this will also add another processing step.

Therefore, methods for preventing clogging of the flow channel and inhibition of immune reactions easily and expediently without preliminary processes being performed have been proposed. An example of such a method is that in which a surfactant is added to a test sample, to chemically destroy the cell walls of blood cell components to hemolyze blood (Japanese Unexamined Patent Publication No. 2007-163182) . Another example of such a method is that in which ultrasonic waves are emitted into a test sample within a flow channel to physically destroy the cell walls of blood cell components to hemolyze blood (Japanese Unexamined Patent Publication No. 2009-207459).

However, in the case that a surfactant is employed as in the method of Japanese Unexamined Patent Publication No. 2007-163182, the addition of the surfactant will change the fluid dynamic properties (viscosity, surface tension, etc.) of the test sample as a whole. The state of flow of the test sample within the flow channel, such as flow speed, will change as a result. Therefore a problem arises that the speed of immune reactions will be difficult to control, as the speed of reactions will be controlled by dispersion.

In addition, in the case that ultrasonic waves are simply emitted as in the method of Japanese Unexamined Patent Publication No. 2009-207459, the acoustic radiation pressure distribution of the ultrasonic waves will vary in an irregular manner depending on locations within the flow channel and time. Accordingly, there is a problem that the hemolysis rate (the percentage of blood cell components of which the cell walls have been destroyed and hemolyzed) of blood cells which have passed through the acoustic field is not uniform among examinations. Meanwhile, there is another problem that it is necessary to emit the ultrasonic waves for long periods of time in order to uniformize hemolysis rates among examinations.

### SUMMARY OF THE INVENTION

The present invention has been developed in view of the foregoing problems. It is an object of the present invention to provide a target substance detecting method and a target substance detecting apparatus that employ a detecting chip equipped with a fine flow channel, and enable examinations, in which clogging of the flow channel and inhibition of immune reactions can be easily and expediently prevented without preliminary processes, to be performed without fluctuations in hemolysis rates.

A target substance detecting method of the present invention that achieves the above objective employs a detecting chip equipped with: a flow channel base having a flow channel, a flow inlet through which liquid samples are caused to flow into the flow channel, and an air opening for causing the liquid samples which have flowed in through the flow inlet to flow into the flow channel; and a detecting portion formed at a predetermined region within the flow channel, and is characterized by comprising:

causing a liquid sample that may contain a target substance and contains cellular non target substances to flow into the flow channel;

causing an ultrasonic wave emitting section provided upstream of the detecting portion to emit ultrasonic waves into the liquid sample from a direction perpendicular to the longitudinal direction of the flow channel such that a standing wave is generated within the flow channel; and

detecting the target substance with an immobilized binding substance, which is immobilized onto the detecting portion, that specifically binds with the target substance.

In the present specification, the term “detecting portion” refers to a location at which the target substance is detected. For example, in the case that an immobilized binding substance (antibodies, for example) that specifically binds with a target substance (antigens, for example) is immobilized on the surface of the wall of the flow channel, the region of the surface of the wall of the flow channel at which the binding substance is immobilized is the detecting portion. The detecting portion may include a metal film or the like that causes surface plasmon to be generated on the surface of the wall of the flow channel.

The expression “direction perpendicular to the longitudinal direction of the flow channel” refers to the height direction or the width direction of the flow channel.

The expression “immobilized binding substance” refers to one of a pair of substances that specifically bind with each other, which is immobilized onto the detecting portion. Note that the expression “pair of substances that specifically bind with each other” refer to pairs of substances that specifically discriminate each other and bind, such as antigens and antibodies, and proteins and cofactors.

It is preferable for the ultrasonic wave emitting section to emit at least two types of ultrasonic waves having different frequencies in a temporally and/or spatially separated manner, such that at least two standing waves which are temporally and/or spatially separated are generated in the flow channel.

In the present specification, the expression “at least two standing waves which are temporally and/or spatially separated” refers to two or more standing waves of different frequencies, which are formed during different periods of time and/or formed at different locations. Note that in the case that standing waves are formed while continuously modulating the frequencies thereof, each of the individual standing waves are designated as a separate standing wave.

The expression “emit at least two types of ultrasonic waves having different frequencies in a temporally and/or spatially separated manner” refers to ultrasonic waves being emitted during different time periods and/or emitted at different locations. Note that in the case that ultrasonic waves are emitted while continuously modulating the frequencies thereof, each of the individual ultrasonic waves are designated as a separate ultrasonic wave.

The ultrasonic wave emitting section may be constituted by a single ultrasonic wave emitting element. In this case, the ultrasonic wave emitting element emits ultrasonic waves while modulating the frequency thereof over time, to emit the at least two ultrasonic waves having different frequencies such that they are temporally separated. It is preferable for the frequency of the ultrasonic waves to be modulated such that the antinode of a standing wave generated by one of the at least two ultrasonic waves is positioned at a node of a standing wave generated by another ultrasonic wave.

In the present specification, the expression “the antinode of a standing wave generated by one of the at least two ultrasonic waves is positioned at a node of a standing wave generated by another ultrasonic wave” refers to a state in which the node of a standing wave is formed at substantially the same location as the antinode of another standing wave, although not simultaneously. This state is a state in which standing waves are formed such that the position of the node of a standing wave and the position of the node of another standing wave do not overlap, taking dispersion of the target substance within a liquid sample into consideration. At least one pair of an antinode and a node of at least one pair of standing waves may be formed at substantially the same location.

Further, the ultrasonic wave emitting section may be constituted by at least two ultrasonic wave emitting elements which are provided along the longitudinal direction of the flow channel. In this case, the at least two ultrasonic wave emitting elements emit ultrasonic waves having different frequencies, to emit the at least two ultrasonic waves having different frequencies such that they are spatially separated. It is preferable for the different frequencies of the ultrasonic waves to be such that the antinode of a standing wave generated by one of the at least two ultrasonic waves corresponds to the position of a node of a standing wave generated by another ultrasonic wave along the longitudinal direction of the flow channel. Further, it is preferable for the frequency of at least one of the ultrasonic waves from among the at least two ultrasonic waves having different frequencies to be modulated.

In the present specification, the expression “the antinode of a standing wave generated by one of the at least two ultrasonic waves corresponds to the position of a node of a standing wave generated by another ultrasonic wave along the longitudinal direction of the flow channel” refers to a state in which the node of a standing wave and the antinode of another standing wave are present along the same flow line. This state is a state in which standing waves are formed such that the position of the node of a standing wave and the position of the node of another standing wave are present along the same flow line, taking dispersion of the target substance within a liquid sample into consideration. In other words, if the width of the flow channel is constant and the sections at which the standing waves are generated viewed in the longitudinal direction of the flow channel, the node of a standing wave and an antinode of another standing wave overlap each other within a range in which dispersion of substances and molecules within the liquid sample can be ignored. At least one pair of an antinode and anode of at least one pair of standing waves may be formed at substantially the same location.

Further, it is preferable for a detecting chip provided with an acoustic matching layer on a wall surface of the flow channel at a region where the standing wave is to be generated to be employed as the detecting chip.

Meanwhile, a target substance detecting apparatus of the present invention is characterized by comprising:

a detecting chip equipped with: a flow channel base having a flow channel, a flow inlet through which liquid samples are caused to flow into the flow channel, and an air opening for causing the liquid samples which have flowed in through the flow inlet to flow into the flow channel; and a detecting portion formed at a predetermined region within the flow channel;

an ultrasonic wave emitting section provided upstream of the detecting portion for emitting ultrasonic waves into the liquid sample from a direction perpendicular to the longitudinal direction of the flow channel; and

an ultrasonic wave controlling section for controlling the ultrasonic wave emitting section such that a standing wave is generated within the flow channel.

It is preferable for the ultrasonic wave emitting section is constituted by a single ultrasonic wave emitting element. In this case, the ultrasonic wave emitting element emits ultrasonic waves while modulating the frequency thereof over time, to emit at least two ultrasonic waves having different frequencies such that they are temporally separated. Alternatively, it is preferable for the ultrasonic wave emitting section to be constituted by at least two ultrasonic wave emitting elements which are provided along the longitudinal direction of the flow channel. In this case, the at least two ultrasonic wave emitting elements emit ultrasonic waves having different frequencies, to emit at least two ultrasonic waves having different frequencies such that they are spatially separated.

The target substance detecting method of the present invention employs the detecting chip equipped with the fine flow channel, causes a liquid sample that may contain a target substance and contains cellular non target substances to flow into the flow channel; and causes an ultrasonic wave emitting section provided upstream of the detecting portion to emit ultrasonic waves into the liquid sample from a direction perpendicular to the longitudinal direction of the flow channel such that a standing wave is generated within the flow channel. Accordingly, cellular non target substances can be physically destroyed, and therefore, clogging of the flow channel and inhibition of immune reactions can be easily and expediently prevented without a preliminary process being performed. In addition, because standing waves are generated within the flow channel, the positions of the antinodes and the nodes are fixed, the acoustic radiation pressure distribution becomes uniform, and examinations can be performed such that fluctuations in hemolysis rates do not occur among examinations. As a result, examinations, in which clogging of the flow channel and inhibition of immune reactions can be easily and expediently prevented without a preliminary process, are enabled to be performed without fluctuations in hemolysis rates.

The target substance detecting apparatus of the present invention is characterized by comprising: a detecting chip equipped with: a flow channel base having a flow channel, a flow inlet through which liquid samples are caused to flow into the flow channel, and an air opening for causing the liquid samples which have flowed in through the flow inlet to flow into the flow channel; and a detecting portion formed at a predetermined region within the flow channel; an ultrasonic wave emitting section provided upstream of the detecting portion for emitting ultrasonic waves into the liquid sample from a direction perpendicular to the longitudinal direction of the flow channel; and an ultrasonic wave controlling section for controlling the ultrasonic wave emitting section such that a standing wave is generated within the flow channel. The ultrasonic wave emitting section emits ultrasonic waves from a direction perpendicular to the longitudinal direction of the flow channel such that a standing wave is generated in the liquid sample within the flow channel. As a result, examinations, in which clogging of the flow channel and inhibition of immune reactions can be easily and expediently prevented without a preliminary process, are enabled to be performed without fluctuations in hemolysis rates, in the same manner as in the target substance detecting method of the present invention.

### BRIEF DESCRIPTION OF THE DRAWINGS

FIG. 1A is a sectional view that schematically illustrates a detecting apparatus which is employed in a target substance detecting method according to a first embodiment of the present invention.

FIG. 1B is a plan view that schematically illustrates a detecting chip which is employed in the target substance detecting method according to the first embodiment of the present invention.

FIG. 1C is a sectional view that schematically illustrates a detecting chip which is employed in the target substance detecting method according to the first embodiment of the present invention.

FIG. 2 is a collection of sectional views that schematically illustrate the steps of an immunological examination performed by the target substance detecting method according to the first embodiment of the present invention using the sandwich method.

FIG. 3A and FIG. 3B are sectional diagrams that schematically illustrate stated in which standing waves are generated by ultrasonic waves having different frequencies during different periods of time in the first embodiment.

FIG. 4 is a sectional view that schematically illustrates a detecting chip which is employed in a target substance detecting method according to a second embodiment of the present invention.

FIG. 5 is a sectional diagram that schematically illustrates a state in which standing waves of different frequencies are generated at different locations in the second embodiment.

FIG. 6 is a sectional view that schematically illustrates a detecting chip which is employed in a target substance detecting method according to a third embodiment of the present invention.

FIG. 7 is a sectional diagram that schematically illustrates a state in which standing waves of different frequencies are generated at different locations in the third embodiment.

FIG. 8 is a sectional diagram that schematically illustrates a state in which standing waves of the same frequency are generated at different locations in the third embodiment.

### DESCRIPTION OF THE PREFERRED EMBODIMENTS

Hereinafter, embodiments of the present invention will be described with reference to the attached drawings. However, the present invention is not limited to the embodiments to be described below. Note that the dimensions of the elements in the drawings differ from the actual dimensions thereof, to facilitate visual understanding.

### First Embodiment of the Target Substance Detecting Method

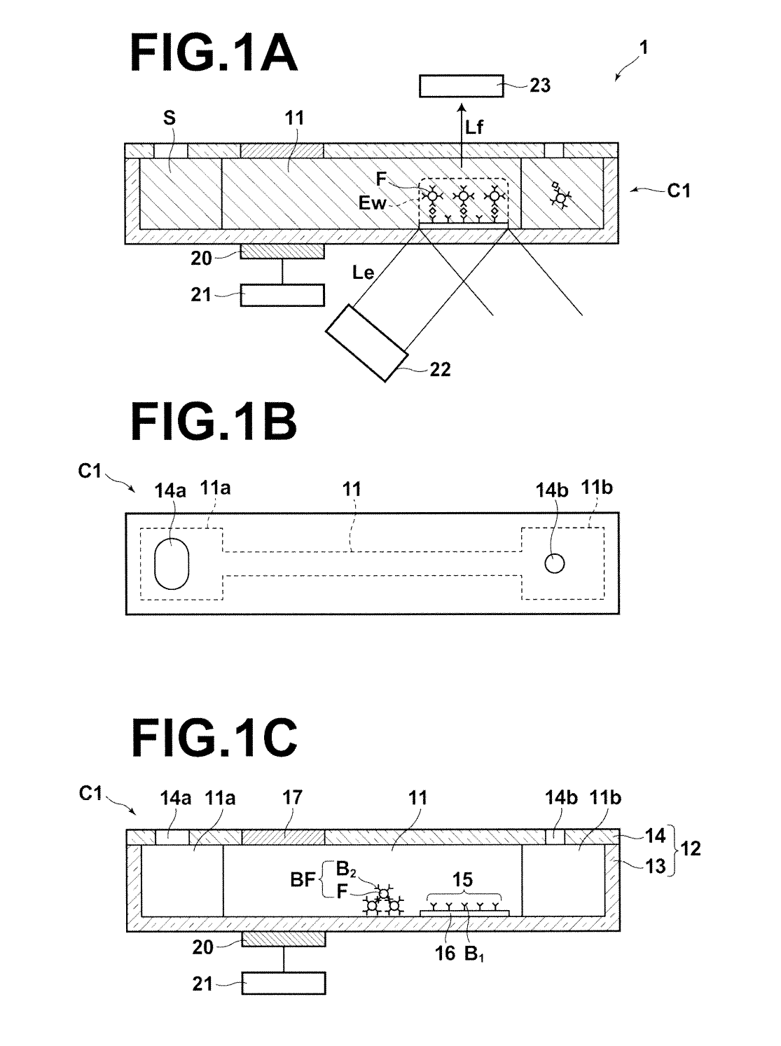

First, a target substance detecting method according to a first embodiment will be described. FIG. 1A is a sectional view that schematically illustrates a detecting apparatus which is employed in a target substance detecting method according to a first embodiment of the present invention. FIG. 1B and FIG. 10 are a plan view and a sectional that schematically illustrate a detecting chip which is employed in the target substance detecting method according to the first embodiment of the present invention, respectively. Note that the first embodiment will be described as a case in which antigens and antibodies are employed as pairs of substances that specifically bind with each other, the detection target substance is an antigen, the binding substance that specifically binds with the detection target substance is an antibody, and analysis is performed by the sandwich method that employs fluorescent labels.

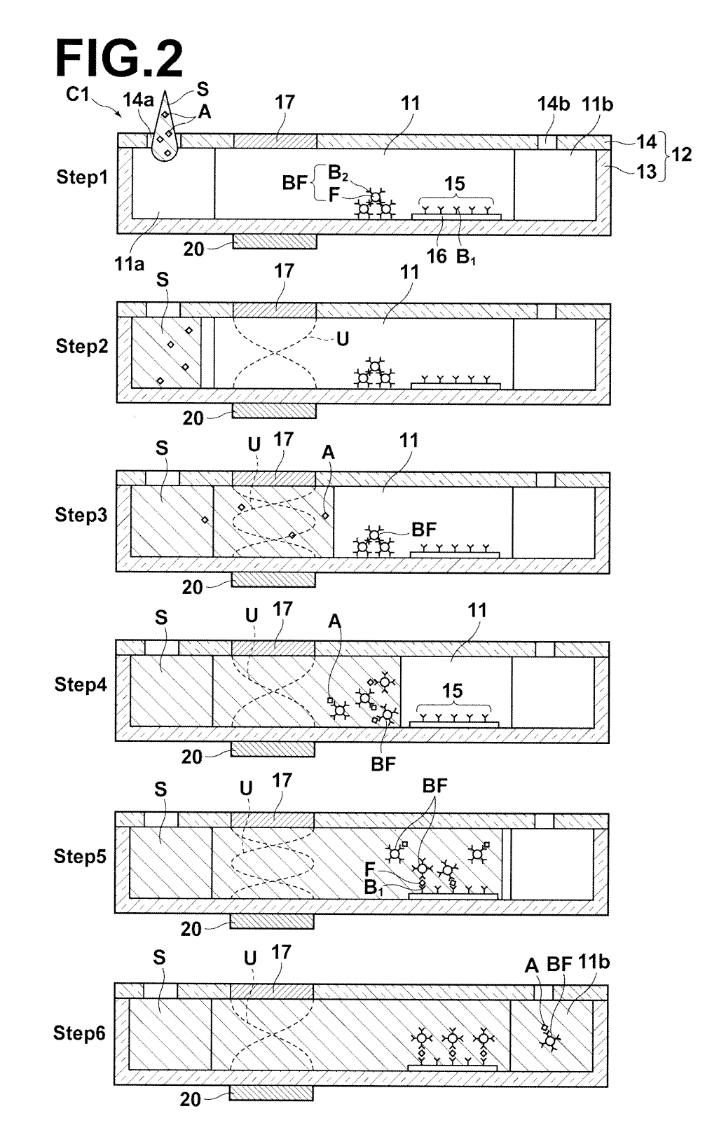

As illustrated in FIG. 2, the target substance detecting method of the first embodiment employs a detecting chip C1, which includes a flow channel base 12 having a flow channel 11, a flow inlet 14a through which a liquid sample is caused to flow into the flow channel 11, and an air opening 14b for causing the liquid sample which has flowed in through the flow inlet 14a to flow into the flow channel 11; and a detecting portion 15 formed at a predetermined region within the flow channel 11. Fluorescent labeling antibodies BF are provided in a dry state upstream of the detecting portion 15. A whole blood sample S that may contain antigens A is caused to flow into the flow channel 11. An ultrasonic wave emitting section 20 provided upstream of the region at which the fluorescent labeling antibodies BF are provided emits ultrasonic waves toward the whole blood sample S within the flow channel 11 from a direction perpendicular to the longitudinal direction of the flow channel 11. The frequency of the ultrasonic waves is periodically modulated such that standing waves U of different frequencies are periodically generated within the flow channel 11. The acoustic radiation pressure at the antinodes of the standing waves U destroys the cell walls of the blood cell components within the whole blood sample S. Thereby, the whole blood sample S is caused to flow through the flow channel 11 without clogging the flow channel 11. The whole blood sample S contacts the fluorescent labeling antibodies BF, the antigens A are labeled by the fluorescent labeling antibodies BF, and combinations of the antigens A and the fluorescent labeling antibodies BF are immobilized onto immobilized antibodies B1 provided on the detecting portion 15. Then, a measuring light beam Le is irradiated, a photodetector 23 detects fluorescence Lf emitted by fluorescent labels F within the combinations, and the presence or absence and/or the amount of the antigens A within the whole blood sample S is detected.

As illustrated in FIG. 1A, a target substance detecting apparatus of the present invention is equipped with: the ultrasonic wave emitting section 20 provided upstream of the detecting portion that emits ultrasonic waves into liquid samples within the flow channel, from a direction perpendicular to the longitudinal direction of the flow channel; a ultrasonic wave control section 21 that controls the ultrasonic wave emitting section such that standing waves are generated within the flow channel; a light source 22; and the photodetector 23.

The detecting chip C1 which is employed in the target substance detecting method and the target substance detecting apparatus is basically constituted by the flow channel base 12 having the flow channel, and the detecting portion 15 within the flow channel.

The flow channel base 12 includes a flow channel member 13 having a U shaped groove, and a lid member 14 that covers the groove. The flow channel base 12 is preferably formed by resin. In the case that the flow channel member 54 is formed by resin, polymethyl methacrylate (PMMA), polycarbonate (PC), non crystalline polyolefin (APO) that includes cycloolefin, polystyrene, and Zeonex™ are particularly preferred.

The flow channel 11 is formed by the lid member 14 being mounted on the flow channel member 13 to cover the U shaped groove formed in the flow channel member 13. In the present specification, the width and the height of the flow channel 11 (both corresponding to a direction perpendicular to the flow direction of sample liquids) are not particularly limited. Clogging occurs conspicuously in cases that the width is 10 mm or less and the height is 2 mm or less, and particularly in cases that the width is 3 mm or less and the height is 500 μm or less. Therefore, the present invention is particularly effective with respect to examinations that employ flow channels with widths and heights in these ranges. As a specific example, the flow channel 11 of the present embodiment has a width of 2 mm and a height of 314 μm. Further, liquid reservoirs 11a and 11b for injection and for discharge are respectively provided at the ends of the flow channel 11.

A metal film 16 that functions as the detecting portion is formed at the predetermined region within the flow channel 11. Thereby, a high sensitivity analysis method that utilizes an enhancing field Ew due to plasmon generated in the metal film 16 can be realized. For example, in the case that the metal film 16 is a film formed by metallic material, the surface plasmon resonance method, the surface plasmon enhanced fluorometry method, or the SPCE (Surface Plasmon Coupled Emission) method may be utilized. In the case that the metal film 16 is a layer formed by fine metal particles, the local surface plasmon resonance method may be utilized. The material of the metal film 16 is not particularly limited. Examples of materials which are desirable from the viewpoint of inducing plasmon include Au, Ag, Cu, Pt, Ni, and Ti. Among these, Au and Ag, which exhibit high electric field enhancing effects, are particularly preferred. It is desirable for the thicknesses of the metal film 16 to be determined such that surface plasmon is strongly excited, taking the material of the metal film 16 and the wavelength of the measuring light beam Le into consideration. For example, in the case that a laser beam having a central wavelength of 780 nm is employed as the measuring light beam Le, and Au is employed as the material of the metal film 16, a favorable thickness of the metal film 16 is 50 nm±5 nm.

Further, a reflecting plate 17 that efficiently reflects the ultrasonic waves is provided within the flow channel 11. The reflecting plate 17 is provided to efficiently reflect the ultrasonic waves (that is, to efficiently cause the standing wave U to be generated) Therefore, the reflecting plate 17 is provided to face the ultrasonic emitting section 20 to be described later. It is preferable for a material that has an acoustic impedance which is greatly different from that of the liquid sample S, in order to increase the reflectance with respect to ultrasonic waves. Examples of such materials include dielectrics such as glass, and metals such as aluminum. The reflecting plate 17 may be embedded into an inner wall of the flow channel 11 as illustrated in FIG. 1C (within the lid member 14 in FIG. 1C) or formed on the surface of an inner wall of flow channel 11. In addition, the flow channel base 12 itself may be the reflecting plate 17, if the acoustic impedance thereof differs greatly from that of the liquid sample. That is, the flow channel base 12 may function as a reflecting plate. However, from the viewpoint of material selection based on the size of acoustic impedance, it is preferable for the reflecting layer 17 to be provided separately, using a material different from that of the flow channel base.

The lid member 14 forms the top surface of the flow channel 11, by being fitted on the flow channel member 13. The flow inlet 14a, which is connected to the liquid reservoir 11a for injecting liquids and functions to inject fluids therein, and an air opening 14b, which is connected to the liquid reservoir lib for discharging liquids and functions to suction air therefrom, are formed in the lid member 14. The lid member 14 is fitted onto the flow channel member 13 by ultrasonic welding or the like after the metal film 16 is formed.

The liquid sample S in the present embodiment is whole blood collected from a human, and the cellular non target substances are the blood cell components included in whole blood. However, the present invention is not limited regarding the liquid sample, and the liquid sample may be blood which is diluted by a diluent. In addition, urine may be collected as a liquid sample instead of blood. That is, the present invention may be applied to remove cellular components such as red blood cells mixed into urine, when detecting 8-OHdG (an oxidant stress marker) within urine.

The fluorescent labeling antibodies BF are provided in a dry state toward the upstream side of the flow channel. The fluorescent labeling antibodies BF are constituted by antibodies B2 and fluorescent labels F that modify the antibodies B2. In the present embodiment, specifically, the fluorescent labeling antibodies BF are constituted by the antibodies B2 (modified binding substance) that specifically bind with the antigens A (target substance) and the fluorescent labels F (labeling substance) that modifies the antibodies B2. The combinations of the fluorescent labeling antibodies BF and the antigens A are formed by the antibodies B2 specifically binding with the antigens A. Therefore, a separate operation for labeling the antigens A is obviated. The fluorescent labels F (labeling substance) is not particularly limited, and fluorescent pigment molecules, quantum dots, etc., may be employed. It is preferable for fluorescent particles (labeling particles) formed by polystyrene particles, for example, to envelop a plurality of fluorescent pigment molecules, quantum dots, etc. in the interiors thereof, to enable fluorescent detection to be performed with high sensitivity. It is preferable for the size of the labeling particles to be within a range from 0.05 μm to 10 mm, and more preferably 0.1 μm to 1 mm, because the capturing forces of standing waves is proportionate to the volumes of substances, and from the viewpoint of dispersion speed. Although labeling is performed within the flow channel 11 after the sample is supplied to the flow channel in the present embodiment, the timing at which the antigens A are labeled is not particularly limited. The antigens A may be caused to react with the labels prior to the sample being supplied to the flow channel 11.

The detecting portion 15 is a region that includes the metal film 16, on which the immobilized antibodies B1 for detecting the antigens A are immobilized. A single detecting portion 15 or a plurality of sensor portions 15 may be provided. In the case that a plurality of sensor portions 15 having different types of immobilized antibodies immobilized thereon are provided, a plurality of types of antigens can be detected, and therefore, multiple item array analysis and measurement becomes possible. The immobilized antibodies B1 are antibodies that specifically bind with the antigens A. The type of antibody is not particularly limited, and may be selected according to detection conditions (particularly the type of antigen the antigen A is). For example, in the case that the antigens A are hCG antigens (molecular weight: 38,000 Da), monoclonal antibodies that specifically bind with the antigens A may be employed as the immobilized antibodies B1. Examples of methods by which the immobilized antibodies B1 may be immobilized onto metal film 16 include physical adsorption, and immobilization by static electricity or by chemical bonds after introducing functional groups such as carboxyl groups, amino groups, and thiol groups onto the acoustic matching layer by surface modifications.

The ultrasonic wave emitting section 20 is provided upstream of the detecting portion 15, and emits ultrasonic waves into the whole blood sample within the flow channel 11 from a direction (from the lower direction within the plane of the drawing sheet of FIG. 1A in the present embodiment) perpendicular to the longitudinal direction of the flow channel 11 such that a standing wave is generated within the flow channel 11. Thereby, a standing wave U is generated between the upper and lower walls of the flow channel 11 of FIG. 1A. The ultrasonic wave emitting section 20 is an ultrasonic wave transducer, for example. Ultrasonic wave transducers are piezoelectric elements formed by piezoelectric ceramics, or by polymeric molecular films such as polyvinyl pyrolidone. PZT-Pb (Zr·Ti) O3 type Soft Material C-82 (by Fuji Ceramics) is a preferred ultrasonic wave transducer. In the present embodiment, the ultrasonic wave emitting section 20 is constituted by a single ultrasonic wave emitting element (an ultrasonic transducer, for example). However, the ultrasonic wave emitting section 20 may be constituted by two or more ultrasonic wave emitting elements, as will be described later. That is, the present embodiment employs a single ultrasonic wave emitting element and modulates the frequency of the emitted ultrasonic waves, to emit ultrasonic waves having different frequencies which are temporally separated. The ultrasonic wave emitting section 20 may be formed as a portion of the surface of the wall of the flow channel such that it directly contacts the liquid sample S within the flow channel. However, taking the fact that the apparatus will be used repeatedly into consideration, it is preferable for the ultrasonic wave emitting section 20 emit ultrasonic waves into the flow channel 11 through the flow channel base 12. The frequency of the ultrasonic waves can be set according to the length of the flow channel 11 in the direction in which the ultrasonic waves are emitted. It is preferable for the frequency to be within a range from 100 kHz to 100 MHz. A frequency of approximately 3 MHz is particularly preferred. The frequency of the ultrasonic waves may be uniform over time. However, it is preferable for the frequency to be temporally and/or periodically modulated by continuously sweeping or intermittently changing the frequency, in order to efficiently destroy the blood cell components (cellular non target substances).

The ultrasonic wave control section 21 controls the ultrasonic wave emitting section 20 such that the standing waves U are generated within the flow channel 11. The ultrasonic wave control section 21 may be a separate unit equipped with a power source, an ultrasonic wave generating circuit, a modulating circuit, and an output circuit. In addition, the ultrasonic wave control section 21 may be further equipped with additional circuits, such as a circuit for freely forming the waveform of the ultrasonic waves as necessary. A multifunction generator WF1974 (by NF Corporation) may be employed as the ultrasonic wave control section 21, for example. The waveform of the drive voltage may be any desired shape, such as a sine wave, a rectangular wave, a triangular wave, and a ramp wave.

The light source 22 is not particularly limited, and may be a laser light source. As described previously, the light source 22 is arranged such that the measuring light beam Le output thereby enters the interface between the flow channel base 12 and the metal film 16 of the sensor chip C1 at a resonance angle that causes total reflection of the measuring light beam Le at the interface, such that surface plasmon resonance occurs at the metal film 16. Note that it is preferable for the measuring light beam Le to be P polarized light, such that surface plasmon can be induced.

The photodetector 23 detects fluorescence Lf generated by the fluorescent labels F. Examples of photodetectors to be employed as the photodetector 23 include: CCD's; PD's (photodiodes); photomultipliers; and c-MOS's. It is preferable for a cooled CCD to be employed from the viewpoint of detection sensitivity.

Hereinafter, the procedures by which an assay is performed according to the sandwich method to detect whether the antigen A, which is a target substance, is included in a whole blood sample S by the target substance detecting method of the present embodiment using the detecting chip C1 will be described with reference to FIG. 2.

Step 1: The sample S, which is the target of inspection, is injected into the detecting chip C1 through the flow inlet 14a.

Step 2: After the sample S is caused to flow into the flow channel, the ultrasonic wave emitting section 20 emits ultrasonic waves in the height direction of the flow channel 11 while modulating the frequency thereof, to generate standing waves U within the flow channel 11.

Step 3: The sample S leaks out into the channel 11 by capillary action. At this time, the sample S passes through the region at which the standing waves U are generated (standing wave region). Mechanical forces of acoustic radiation pressure of the standing waves U destroy the blood cell components. Meanwhile, the antigens A are small compared to the blood cell components, therefore are not influenced by the acoustic radiation pressure, and pass through the standing wave region as they are. A pump may be connected to the air opening 14b, and the sample S may be caused to flow by suctioning and extruding operations of the pump, in order to expedite reactions and to shorten detection time.

Step 4: the sample S, which has passed through the standing wave region, and the fluorescent labeling antibodies BF (the fluorescent particles F modified with the antibodies B2) mix, the antigens A within the sample S bind with the labeling secondary antibodies BF, and the sample flows further along the flow channel 11.

Step 5: the sample S gradually flows along the flow channel 11 toward the air opening 14b, and the antigens A, which are bound to the labeling secondary antibodies BF, bind with the immobilized antibodies B1, which are immobilized onto the detecting portion 15. So called sandwich configurations, in which the antigens A are sandwiched between the immobilized antibodies B1 and the labeling antibodies BF, are formed.

Step 6: Even in the case that the labeling antibodies BF which were not immobilized onto the detecting portion 15 remain on the detecting portion 15, the following sample S functions as a cleansing agent that washes the labeling antibodies BF, which are floating or non specifically adsorbed onto the detecting portion 15, away. The sample S which has passed over the detecting portion 15 is collected in a waste liquid reservoir lib.

The antigens A can be detected by irradiating the measuring light beam Le onto the detecting portion 15 and then detecting fluorescence Lf emitted by the fluorescent labels F after performing the above steps.

Hereinafter, the advantageous effects of the first embodiment of the present invention will be described in detail.

As described previously, the present invention is characterized by the ultrasonic wave emitting section 20 generating standing waves within the flow channel in the target substance detecting method that employs the detecting chip C equipped with the fine flow channel 11. This feature enables examinations to be performed, in which clogging of the flow channel 11 and inhibition of immune reactions can be easily and expediently prevented without a preliminary process, to be performed with respect to liquid samples S that contain target substances and cellular non target substances without fluctuations in hemolysis rates.

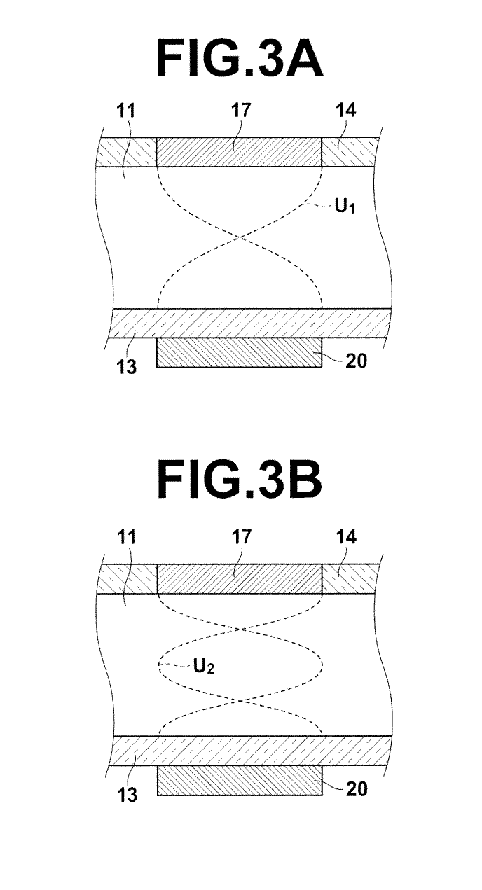

For example, a case will be considered in which the ultrasonic wave emitting section 20 emits ultrasonic waves having frequencies of 2.5 MHz and 5.0 MHz into the flow channel 11 of the detecting chip C1, which has a depth of approximately 314 μm. The flow channel 11 is filled with the whole blood sample S, and therefore the speed of sound within the flow channel 11 is 1570 m/sec. in this case, standing waves U1 and U2 as illustrated in FIG. 3A and FIG. 3B, respectively, are generated. The standing wave U1 generated by the ultrasonic waves having the frequency of 2.5 MHz has antinodes at the wall surfaces of the flow channel 11 and a single node within the flow channel 11, as illustrated in FIG. 3A. The standing wave U2 generated by the ultrasonic waves having the frequency of 5.0 MHz has antinodes at the wall surfaces of the flow channel 11 and two nodes within the flow channel 11, as illustrated in FIG. 3B. That is, the distance between the antinodes of the standing wave U1 is 314 μmm, and the distance between the antinodes of the standing wave U2 is 157 μm. When the whole blood sample S flows into the flow channel 11, the blood cell components pass through the standing wave region. The acoustic radiation pressure distributions of the standing waves U are fixed, and therefore a stable hemolysis rate can be obtained, although there are differences in how easily the blood cell components are destroyed at the node positions and the antinode positions. Further, the positions of the antinodes and the positions of the nodes are periodically switched by modulating the frequency as described above. Therefore, blood cell components which are concentrated at the position of the node of one of the standing waves can be destroyed by the acoustic radiation pressure of the antinode of the other wave, enabling efficient hemolysis. This is because capturing forces operate on substances in the vicinity of the nodes of standing waves to draw them toward the nodes.

The capturing forces are forces that operate toward the nearest node. The intensity F₀ of the capturing forces is proportionate to the volume V of a substance M, the frequency f of a sound wave, etc., as shown in Formula (1) below.

$${F}_{0} ( z ) = \frac{5 π}{4 {ρ}_{0} {c}_{0}^{3}} {VP}_{0}^{2} f sin ( 2 π \frac{z}{λ / 2} )$$

wherein F₀ represents the intensity of the capturing forces, z represents the distance from the end of the standing wave U as illustrated in FIG. 5, ρ₀ the density of a medium, c₀ represents the speed of sound in a medium, V represents the volume of a substance M, P₀ represents acoustic pressure, f represents the frequency of the standing wave, and λ represents the wavelength of the standing wave. Alternatively, it can be said that the intensity of the capturing force is proportional to the slope of acoustic radiation pressure potential. Accordingly, the capturing force F₀ is greatest with respect to substances M which are close to the positions of antinodes of standing waves U, and substances move only a slight amount at the positions of nodes. Here, the acoustic radiation pressure potential refers to the mechanical potential within the acoustic field of standing waves.

Blood cell components which are not hemolyzed inhibit immunological reactions, or influence signal noise by scattering signal light during optical measurements and by adsorbing to the detecting portion, and reduce the reliability of examinations. However, higher and more uniform hemolysis rates can be realized compared to the conventional case in which propagating waves are emitted, by generating standing waves while periodically switching the frequency thereof and utilizing the nodes and antinodes of the standing waves having different frequencies as in the first embodiment. This is because standing waves have higher acoustic radiation pressure (the amplitude of the antinodes of standing waves are twice those of propagating waves) than those of propagating waves having a uniform distribution.

As described above, the target substance detecting method of the first embodiment employs the detecting chip C1 equipped with the fine flow channel, causes a liquid sample that may contain a target substance and contains cellular non target substances to flow into the flow channel; and causes an ultrasonic wave emitting section provided upstream of the detecting portion to emit ultrasonic waves into the liquid sample from a direction perpendicular to the longitudinal direction of the flow channel such that a standing wave is generated within the flow channel. Accordingly, cellular non target substances can be physically destroyed, and therefore, clogging of the flow channel and inhibition of immune reactions can be easily and expediently prevented without a preliminary process being performed. In addition, because standing waves are generated within the flow channel, the positions of the antinodes and the nodes are spatially fixed, the acoustic radiation pressure distribution becomes uniform, and examinations can be performed such that fluctuations in hemolysis rates do not occur among examinations. As a result, examinations, in which clogging of the flow channel and inhibition of immune reactions can be easily and expediently prevented without a preliminary process, are enabled to be performed without fluctuations in hemolysis rates.

Further, the target substance detecting method of the first embodiment utilizes standing waves, which have a greater acoustic radiation pressure than propagating waves. Therefore, destruction of the cellular non target substances can be easily executed compared to cases in which propagating waves are employed.

Still further, the first embodiment generates the standing waves while periodically switching the frequency thereof, to cause the ultrasonic wave emitting section to emit ultrasonic waves of two or more different frequencies to generate two or more standing waves, which are temporally separated, within the flow channel. Therefore, blood cell components which are concentrated at the position of the node of one of the standing waves can be destroyed by the acoustic radiation pressure of the antinode of the other wave, enabling efficient hemolysis.

### Design Modifications to the First Embodiment

In the embodiment described above, fluorescent labels are employed as the labels for the antigens. Alternatively, other photoresponsive labels (such as phosphorous labels, and scattered light labels) may be employed as the labels. In addition, the target substance detecting method of the first embodiment may be combined with various other types of immune measurement methods, such as the radioimmunoassay method (RIA) that employs radioactive isotopes, the enzyme immunoassay method (EIA) that employs enzymes, and the chemiluminescent enzyme immunoassay method (CLEIA).

In addition, the above embodiment was described as cases in which the sandwich method was employed. However, the present invention may also be applied to the competition method. In this case, modified antibodies (modified binding substance) and fluorescent particles that modify the modified antibodies (modified binding substance) that specifically bind with the immobilized antibodies (immobilized binding substance) in a competitive manner with the antigens (target substance) are employed as the fluorescent labeling antibodies.

Note that the frequencies of ultrasonic waves are determined based on the designed heights (depths) of the flow channels in the present invention. In actuality, however, shifting in resonance frequencies occur due to shifting of the heights of flow channels from the designs thereof, and by shifting in adhesion positions between the ultrasonic wave emitting section and the flow channel base. Therefore, it is necessary to sweep the frequency of voltages to be applied to the ultrasonic wave emitting section, to adjust the frequency of the ultrasonic wave to that which can most efficiently concentrate and hemolyze blood cells.

### Second Embodiment of the Target Substance Detecting Method

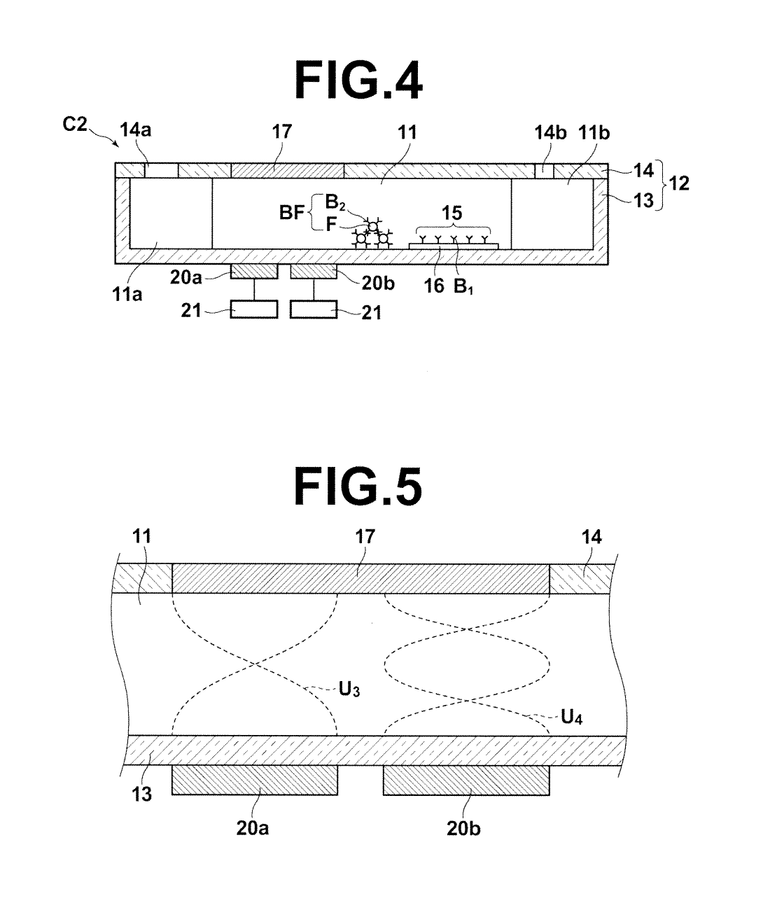

Next, a target substance detecting method according to a second embodiment of the present invention will be described. FIG. 4 is a sectional view that schematically illustrates a detecting chip C2 which is employed in the target substance detecting method of the second embodiment. Note that the second embodiment will also be described as a case in which antigens and antibodies are employed as pairs of substances that specifically bind with each other, the detection target substance is an antigen, the binding substance that specifically binds with the detection target substance is an antibody, and analysis is performed by the sandwich method that employs fluorescent labels.

The target substance detecting method of the second embodiment differs from the first embodiment in that two ultrasonic wave emitting elements 20a and 20b are provided along the flow channel 11 as the ultrasonic wave emitting section. The ultrasonic wave emitting elements 20a and 20b emit ultrasonic waves of different frequencies to generate standing waves U3 and U4 within the flow channel 11. Accordingly, detailed descriptions of the other elements which are the same as those of the first embodiment will be omitted insofar as they are not particularly necessary.

The target substance detecting method of the second embodiment employs a detecting chip C2, which includes a flow channel base 12 having a flow channel 11, a flow inlet 14a through which a liquid sample is caused to flow into the flow channel 11, and an air opening 14b for causing the liquid sample which has flowed in through the flow inlet 14a to flow into the flow channel 11; and a detecting portion 15 formed at a predetermined region within the flow channel 11. Fluorescent labeling antibodies BF are provided in a dry state upstream of the detecting portion 15. A whole blood sample S that may contain antigens A is caused to flow into the flow channel 11. An ultrasonic wave emitting section constituted by the ultrasonic wave emitting elements 20a and 20b is provided upstream of the region at which the fluorescent labeling antibodies BF are provided. The ultrasonic wave emitting elements 20a and 20b emit ultrasonic waves of different frequencies toward the whole blood sample S within the flow channel 11 from a direction perpendicular to the longitudinal direction of the flow channel 11. The ultrasonic generate standing waves U3 and U4 within the flow channel 11. The acoustic radiation pressure at the antinodes of the standing waves U3 and U4 destroys the cell walls of the blood cell components within the whole blood sample S. Thereby, the whole blood sample S is caused to flow through the flow channel 11 without clogging the flow channel 11. The whole blood sample S contacts the fluorescent labeling antibodies BF, the antigens A are labeled by the fluorescent labeling antibodies BF, and combinations of the antigens A and the fluorescent labeling antibodies BF are immobilized onto immobilized antibodies B1 provided on the detecting portion 15. Then, a measuring light beam Le is irradiated, a photodetector 23 detects fluorescence Lf emitted by fluorescent labels F within the combinations, and the presence or absence and/or the amount of the antigens A within the whole blood sample S is detected.

The ultrasonic wave emitting section of the second embodiment is constituted by the two ultrasonic wave emitting elements 20a and 20b which are provided along the longitudinal direction of the flow channel 11. That is, in the second embodiment, separate ultrasonic wave emitting elements are employed to emit ultrasonic waves having different frequencies which are spatially separated. In the second embodiment, ultrasonic waves having different frequencies can be emitted from each of the ultrasonic wave emitting elements without modulating the frequencies. However, the frequencies may be modulated, from the viewpoint of performing hemolysis more efficiently.

FIG. 5 is a sectional diagram that schematically illustrates a state in which standing waves are generated using the detecting chip C2 and the ultrasonic wave emitting section described above. The position of an antinode of the standing wave U4 corresponds to the position of the node of the other standing wave U3 along the longitudinal direction of the flow channel 11. Thereby, blood cell components which are concentrated at the node of the standing wave U3, which is upstream of the standing wave U4, can be destroyed by the acoustic radiation pressure of the antinode of the standing wave U4. In this manner, cellular non target substances can be efficiently destroyed. In this case, in order to increase the hemolysis rate, a bipolar power source such as HSA4101 (by NF Circuit Design Block) may be employed for the downstream ultrasonic wave emitting element, to amplify voltages prior to application thereof.

In the second embodiment, the blood cell components pass through the two standing wave regions when the whole blood sample is caused to flow through the flow channel 11. Because the acoustic radiation pressure distribution is spatially fixed for the standing waves, stable hemolysis rates can be obtained, in the same manner as in the first embodiment. Further, the two ultrasonic wave emitting elements 20a and 20b are provided such that the position of an antinode of a standing wave corresponds to the position of a node of the other standing wave along the longitudinal direction of the flow channel. Therefore, blood cell components which are concentrated at the position of the node of the standing waves U3 can be destroyed by the acoustic radiation pressure of the antinode of the standing wave U4, enabling efficient hemolysis.

As described above, the target substance detecting method of the second embodiment employs a detecting chip equipped with a fine flow channel, supplies the liquid sample that may contain a target substance and contains cellular non target substances into the flow channel, and emits ultrasonic waves into the liquid sample within the flow channel from a direction perpendicular to the longitudinal direction of the flow channel using the ultrasonic wave emitting section provided upstream of the detecting portion to generate standing waves in the flow channel, in the same manner as the first embodiment. Accordingly, the second embodiment exhibits the same advantageous effects as those of the first embodiment.

Further, the target substance detecting method of the second embodiment utilizes standing waves, which have a greater acoustic radiation pressure than propagating waves. Therefore, the second embodiment exhibits the same advantageous effects as those of the first embodiment.

Still further, the second embodiment generates the standing waves using the two or more ultrasonic wave emitting elements, to cause the ultrasonic wave emitting section to emit two or more ultrasonic waves of two or more different frequencies to generate two or more standing waves, which are temporally and/or spatially separated, within the flow channel. Therefore, blood cell components which are concentrated at the position of the node of one of the standing waves can be destroyed by the acoustic radiation pressure of the antinode of the other wave, enabling efficient hemolysis.

### Third Embodiment of the Target Substance Detecting Method

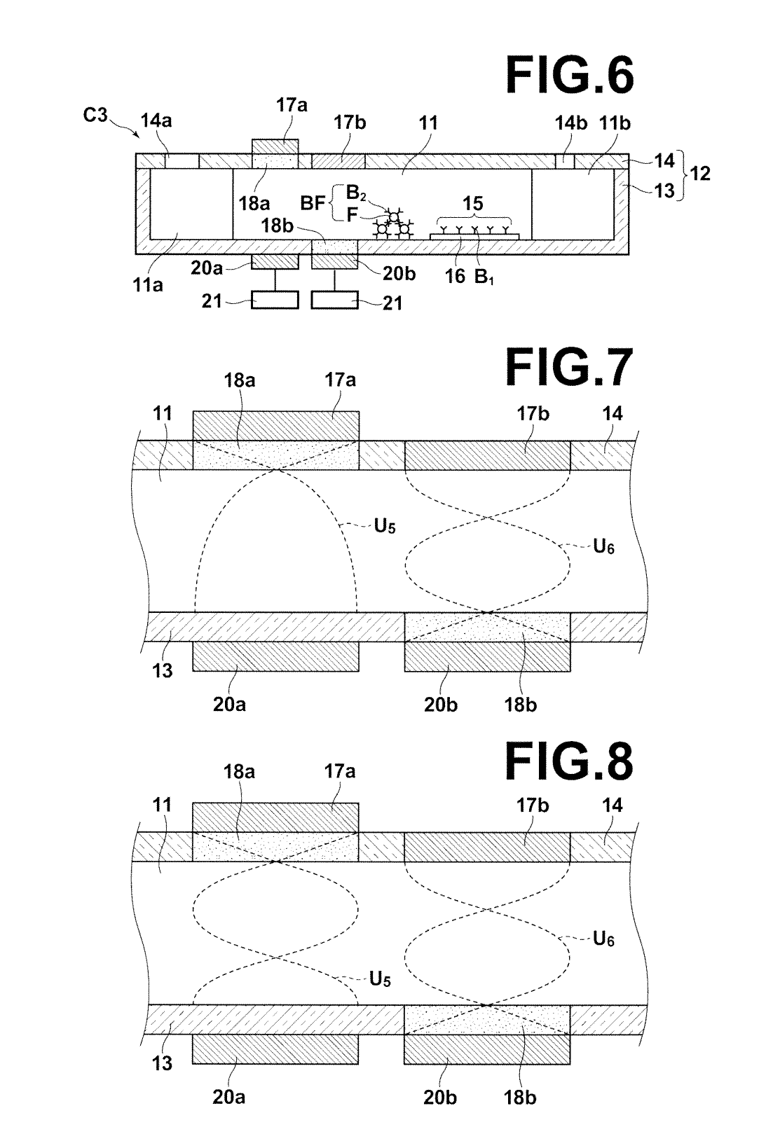

Next, a target substance detecting method according to a third embodiment of the present invention will be described. FIG. 6 is a sectional view that schematically illustrates a detecting chip C3 which is employed in the target substance detecting method of the second embodiment. Note that the third embodiment will also be described as a case in which antigens and antibodies are employed as pairs of substances that specifically bind with each other, the detection target substance is an antigen, the binding substance that specifically binds with the detection target substance is an antibody, and analysis is performed by the sandwich method that employs fluorescent labels.

The target substance detecting method of the third embodiment differs from the second embodiment in that a first acoustic matching layer 18a and a first reflecting plate 17a are provided in the flow channel so as to face the ultrasonic wave emitting element 20a, a second acoustic matching layer 18b is provided in the flow channel so as to face the ultrasonic wave emitting element 20b, and a second reflecting plate 17b is provided in the flow channel so as to face the second acoustic matching layer 18b. Accordingly, detailed descriptions of the other elements which are the same as those of the first embodiment will be omitted insofar as they are not particularly necessary.

The target substance detecting method of the third embodiment employs a detecting chip C3, which includes a flow channel base 12 having a flow channel 11, a flow inlet 14a through which a liquid sample is caused to flow into the flow channel 11, and an air opening 14b for causing the liquid sample which has flowed in through the flow inlet 14a to flow into the flow channel 11; and a detecting portion 15 formed at a predetermined region within the flow channel 11. Fluorescent labeling antibodies BF are provided in a dry state upstream of the detecting portion 15. The detecting chip C3 also includes the first and second reflecting plates 17a and 17b, and the acoustic matching layers 18a and 18b. A whole blood sample S that may contain antigens A is caused to flow into the flow channel 11. An ultrasonic wave emitting section constituted by the ultrasonic wave emitting elements 20a and 20b is provided upstream of the region at which the fluorescent labeling antibodies BF are provided. The ultrasonic wave emitting elements 20a and 20b emit ultrasonic waves of different frequencies toward the whole blood sample S within the flow channel 11 from a direction perpendicular to the longitudinal direction of the flow channel 11. The ultrasonic generate standing waves U5 and U6 within the flow channel 11. The acoustic radiation pressure at the antinodes of the standing waves U3 and U4 destroys the cell walls of the blood cell components within the whole blood sample S. Thereby, the whole blood sample S is caused to flow through the flow channel 11 without clogging the flow channel 11. The whole blood sample S contacts the fluorescent labeling antibodies BF, the antigens A are labeled by the fluorescent labeling antibodies BF, and combinations of the antigens A and the fluorescent labeling antibodies BF are immobilized onto immobilized antibodies B1 provided on the detecting portion 15. Then, a measuring light beam Le is irradiated, a photodetector 23 detects fluorescence Lf emitted by fluorescent labels F within the combinations, and the presence or absence and/or the amount of the antigens A within the whole blood sample S is detected.

The ultrasonic wave emitting section of the third embodiment is constituted by the two ultrasonic wave emitting elements 20a and 20b which are provided along the longitudinal direction of the flow channel 11, in the same manner as in the second embodiment. That is, in the second embodiment, separate ultrasonic wave emitting elements are employed to emit ultrasonic waves having different frequencies which are spatially separated.

The detecting chip C3 of the third embodiment is equipped with the first acoustic matching layer 18a and the first reflecting plate 17a are provided on the flow channel base 12 at the side of the flow channel opposite the ultrasonic wave emitting element 20a so as to face the portion of the flow channel base 12 that the ultrasonic wave emitting element 20a is in contact with. Further, the detecting chip C3 is equipped with the second acoustic matching layer 18b, embedded in the flow channel base 12 at the portion that the ultrasonic wave emitting element 20b is in contact with, and the second reflecting plate 17b provided on the flow channel base 12 at the side of the flow channel opposite the second acoustic matching layer 18b so as to face the second acoustic matching layer 18b. More specifically, the first acoustic matching layer 18a is embedded in the portion of the lid member 14 opposite the portion of the flow channel member 13 that the ultrasonic wave emitting element 20a is in contact with. The first reflecting plate 17a is formed so as to contact the first acoustic matching layer 18a at the side thereof opposite the flow channel. In addition, the second acoustic matching layer 18b is embedded in the portion of the flow channel member 13 that the ultrasonic wave emitting element 20b is in contact with, and the second reflecting plate 17b is embedded in the portion of the lid member 14 at the opposite side of the flow channel so as to face the second acoustic matching layer 18b. The details of the reflecting plates 17a and 17b are the same as those of the reflecting plate 17 of the first embodiment.

The acoustic matching layers 18a and 18b are layers that function to match the acoustic impedance (Z: Z=c (speed of sound within a substance)×ρ (the density of the substance)) of the flow channel and the liquid sample S which is supplied into the flow channel. Therefore, the acoustic matching layers 18a and 18b are formed by a material with an acoustic impedance equivalent to the acoustic impedance of the liquid sample S. The material of the acoustic matching layers 18a and 18b is not particularly limited. Commonly, the liquid sample S in cases that biological substances are analyzed is water (Z=1.48×10⁶ N·s·m⁻³ (at room temperature)). Therefore, dielectric materials such as polymers may be employed as the material of the acoustic matching layers 18a and 18b. Soft polyethylene (Z=1.75×10⁶ N·s·m⁻³ (at room temperature)) and rubber materials are preferable. Silicone rubbers such as PDMS (polydimethylsiloxane), natural rubber (Z=1.50×10⁶ N·s·m⁻³ (at room temperature)), and styrene-butadiene rubber (Z=1.76×10⁶ N·s·m⁻³ (at room temperature)) are more preferable. PDMS (Z=1.06×10⁶ N·s·m⁻³ (at room temperature)) is particularly preferable, from the viewpoint of ease in shaping and controlling the thickness of the layer. In the case that the acoustic matching layers are provided, the nodes of standing waves can be positioned at the interfaces (matching interfaces) between the acoustic matching layers and the flow channel (or the liquid sample), by adjusting frequencies. The acoustic matching layers 18a and 18b may be embedded such that they become portions of the inner walls of the flow channel base 12 as illustrated in FIG. 6. Alternatively, they may be formed on the surfaces of the inner walls of the flow channel base 12.

The acoustic matching layers 18a and 18b are provided in the third embodiment. Therefore, the nodes of the standing waves can be positioned at the matching interfaces, as illustrated in FIG. 7. Accordingly, the node of the standing wave U5 generated by the upstream. ultrasonic wave emitting element 20a at the matching interface and the antinode of the standing wave U6 generated by the downstream ultrasonic wave emitting element 20b at the matching interface correspond along the longitudinal direction of the flow channel. Therefore, blood cell components which are concentrated at the node of the standing wave U5, which is upstream of the standing wave U6, can be destroyed by the acoustic radiation pressure of the antinode of the standing wave U6. Efficient hemolysis can be performed in this manner.

In addition, the second acoustic matching layer 18b is provided at the surface of the wall within the standing wave region formed by the downstream ultrasonic wave emitting element 20b at a position (the wall surface on the same flow line as the detecting portion 15) corresponding to the detecting portion 15 along the longitudinal direction of the flow channel. Therefore, the aforementioned capturing forces concentrate the fluorescent labeling antibodies BF which have captured the antigens on a flow line that continues to the vicinity of the detecting portion 15. Accordingly, highly sensitive detection is enabled.

As described above, the target substance detecting method of the third embodiment employs a detecting chip equipped with a fine flow channel, supplies the liquid sample that may contain a target substance and contains cellular non target substances into the flow channel, and emits ultrasonic waves into the liquid sample within the flow channel from a direction perpendicular to the longitudinal direction of the flow channel using the ultrasonic wave emitting section provided upstream of the detecting portion to generate standing waves in the flow channel, in the same manner as the first embodiment. Accordingly, the second embodiment exhibits the same advantageous effects as those of the first embodiment.

In addition, the target substance detecting method of the third embodiment utilizes standing waves, which have a greater acoustic radiation pressure than propagating waves. Therefore, the second embodiment exhibits the same advantageous effects as those of the first embodiment.

Further, the third embodiment generates the standing waves using the two or more ultrasonic wave emitting elements, to cause the ultrasonic wave emitting section to emit two or more ultrasonic waves of two or more different frequencies to generate two or more standing waves, which are temporally and/or spatially separated, within the flow channel. Therefore, the second embodiment exhibits the same advantageous effects as those of the second embodiment.

Still further, the acoustic matching layer is provided at the wall surface of the flow channel in the region that a standing wave is generated at a position that corresponds to the detecting portion along the longitudinal direction of the flow channel in the third embodiment. Therefore, the target substance can be concentrated on a flow line that continues to the vicinity of the detecting portion. Accordingly, highly sensitive detection is enabled.

### Design Modifications to the Third Embodiment

The third embodiment was described as a case in which two ultrasonic wave emitting elements are driven so as to emit two or more ultrasonic waves such that they are temporally and/or spatially separated. However, the present invention is not limited to such a case. In the detecting chip having the configuration of the third embodiment, ultrasonic waves of the same frequency may be emitted. For example, in the case that the thicknesses of the acoustic matching layers 18a and 18b are the same, the standing waves U5 and U6 will be of shapes which are inverted with respect to each other as illustrated in FIG. 8. Therefore, even if ultrasonic waves of the same frequency are emitted, the position of the antinode of a standing wave generated by one of the ultrasonic waves will correspond to the node of a standing wave generated by another of the ultrasonic waves along the longitudinal direction of the flow channel.

What is claimed is:

1. A target substance detecting method that employs a detecting chip equipped with: a flow channel base having a flow channel, a flow inlet through which liquid samples are caused to flow into the flow channel, and an air opening for causing the liquid samples which have flowed in through the flow inlet to flow into the flow channel; and a detecting portion formed at a predetermined region within the flow channel, comprising:
causing a liquid sample that may contain a target substance and contains cellular non target substances to flow into the flow channel;
causing an ultrasonic wave emitting section provided upstream of the detecting portion to emit ultrasonic waves into the liquid sample from a direction perpendicular to the longitudinal direction of the flow channel such that a standing wave is generated within the flow channel; and
detecting the target substance with an immobilized binding substance, which is immobilized onto the detecting portion, that specifically binds with the target substance.

2. A target substance detecting method as defined in claim 1, wherein:
the ultrasonic wave emitting section emits at least two types of ultrasonic waves having different frequencies in a temporally and/or spatially separated manner, such that at least two standing waves which are temporally and/or spatially separated are generated in the flow channel.

3. A target substance detecting method as defined in claim 2, wherein:
the ultrasonic wave emitting section is constituted by a single ultrasonic wave emitting element; and
the ultrasonic wave emitting element emits ultrasonic waves while modulating the frequency thereof over time, to emit the at least two ultrasonic waves having different frequencies such that they are temporally separated.

4. A target substance detecting method as defined in claim 3, wherein:
the frequency of the ultrasonic waves are modulated such that the antinode of a standing wave generated by one of the at least two ultrasonic waves is positioned at a node of a standing wave generated by another ultrasonic wave.

5. A target substance detecting method as defined in claim 2, wherein:
the ultrasonic wave emitting section is constituted by at least two ultrasonic wave emitting elements which are provided along the longitudinal direction of the flow channel; and
the at least two ultrasonic wave emitting elements emit ultrasonic waves having different frequencies, to emit the at least two ultrasonic waves having different frequencies such that they are spatially separated.

6. A target substance detecting method as defined in claim 5, wherein:
the different frequencies of the ultrasonic waves are such that the antinode of a standing wave generated by one of the at least two ultrasonic waves corresponds to the position of a node of a standing wave generated by another ultrasonic wave along the longitudinal direction of the flow channel.

7. A target substance detecting method as defined in claim 5, wherein:
the frequency of at least one of the ultrasonic waves from among the at least two ultrasonic waves having different frequencies is modulated.

8. A target substance detecting method as defined in claim 1, wherein:
a detecting chip provided with an acoustic matching layer on a wall surface of the flow channel at a region where the standing wave is to be generated is employed as the detecting chip.

9. A target substance detecting apparatus, comprising:
a detecting chip equipped with: a flow channel base having a flow channel, a flow inlet through which liquid samples are caused to flow into the flow channel, and an air opening for causing the liquid samples which have flowed in through the flow inlet to flow into the flow channel; and a detecting portion formed at a predetermined region within the flow channel;
an ultrasonic wave emitting section provided upstream of the detecting portion for emitting ultrasonic waves into the liquid sample from a direction perpendicular to the longitudinal direction of the flow channel; and
an ultrasonic wave controlling section for controlling the ultrasonic wave emitting section such that a standing wave is generated within the flow channel.

10. A target substance detecting apparatus as defined in claim 9, wherein:
the ultrasonic wave emitting section is constituted by a single ultrasonic wave emitting element; and
the ultrasonic wave emitting element emits ultrasonic waves while modulating the frequency thereof over time, to emit at least two ultrasonic waves having different frequencies such that they are temporally separated.

11. A target substance detecting apparatus as defined in claim 9, wherein:
the ultrasonic wave emitting section is constituted by at least two ultrasonic wave emitting elements which are provided along the longitudinal direction of the flow channel; and
the at least two ultrasonic wave emitting elements emit ultrasonic waves having different frequencies, to emit at least two ultrasonic waves having different frequencies such that they are spatially separated.
